(19) United States
(12) Patent Application Publication
(10) Pub. No.: US 2024/0399106 A1
(43) Pub. Date: Dec. 5, 2024

(54) Catheter with Pressure Sensor

(71) Applicant: Straub Medical AG, 7323 Wangs (CH)

(72) Inventors: Fabian Haller, Wangs (CH); Bruno Bahnmueller, Wangs (CH)

(21) Appl. No.: 18/699,362

(22) PCT Filed: Nov. 4, 2021
(86) PCT No.: PCT/EP2021/080637

§ 371 Date: Apr. 8, 2024

(51) Int. Cl. A61M 25/00

(52) U.S. Cl. CPC A61M 25/0054; A61M 2025/0001; A61M 2205/18

(57) ABSTRACT

The present disclosure is directed to a catheter comprising a bendable catheter wall defining a substantially central interior lumen of the catheter, the catheter wall including a sealed chamber accommodating a fluid, the chamber substantially neighbouring the interior lumen and structured to be responsive to kinking of the catheter wall, the catheter further comprising a pressure sensor in fluid communication with the fluid, the pressure sensor configured to measure a pressure of the fluid, the pressure sensor further comprising an interface for communicating the measured pressure to a control unit, the catheter configured to allow for measurement of a change of the pressure in the fluid in response to kinking of the catheter.

### TECHNICAL FIELD

The present disclosure refers to a catheter. Specifically, vascular applications, i.e. vascular catheters, for intravascular guidance, are contemplated. Generally, a catheter in terms of the present disclosure may include an introducer sheath, a guiding sheath or a support catheter. An example is a catheter for aspiration, fragmentation and/or removal of removable material, such as thrombi and emboli, from blood vessels. However, further applications besides atherectomy and thrombectomy are possible, too.

### BACKGROUND

Catheters including pressure sensors are known, such as from EP 1 970 001 A1. The catheter wall includes piezoelectric pressure sensors. Multiple sensors are provided, specifically along the longitudinal axis of the catheter and circumferentially along the catheter wall. Each pressure sensor includes a conductor for sending a voltage signal to a measuring means.

Such catheters allow for measurement of the pressure (only) at locations where the dedicated pressure sensor is provided. Therefore, along the axial direction, a plurality of pressure sensors may be provided, so as to cover a reasonable length of the catheter in the axial direction by way of the measurement of the pressure.

More efficient measurement of the pressure may be desired, preferably without compromising accuracy of the measurement.

### SUMMARY

The present disclosure provides a catheter comprising a bendable/flexible catheter wall defining a (substantially central) interior lumen of the catheter, the catheter wall including a self-contained/sealed chamber accommodating fluid, the chamber separate(d) from the interior lumen and structured to be deformable in response to kinking of at least a part of the catheter wall, the catheter further comprising a pressure sensor in communication with the fluid, the pressure sensor configured to measure a (relative) pressure (change/increase) in/of the fluid, the pressure sensor further comprising an interface for communicating the measured pressure (change) to a control unit, the catheter configured such that kinking of at least a part of the catheter wall is detectable by way of a pressure change (increase) of the fluid in the chamber.

The catheter wall is bendable, i.e. flexible. The chamber is configured such that it is responsive to kinking of the catheter wall in that the chamber, specifically the chamber walls, are sufficiently flexible, so as to be able to respond to kinking of the catheter wall, i.e. to react by way of deformation of the chamber walls in response to kinking of the catheter wall. In particular, the chamber is deformable/compressible upon kinking of the catheter wall. In other words, the catheter (wall) may collapse and result in a kink being formed.

The catheter wall is flexible enough so as to allow for bending of the catheter, but is strong enough so that the chamber/catheter wall does not give in in response to a pressure increase within the chamber. (Bending usually does not cause a (substantial) pressure increase in the chamber).

For example, a thickness of at least 0.05 mm for the catheter wall may be appropriate.

The self-contained/sealed chamber accommodating fluid may be described as a chamber which is fluidly isolated. Upon kinking of the catheter (which may happen in abnormal conditions), the volume housed by the chamber is reduced. As the chamber is sealed, this results in the volume (i.e. the fluid) being compressed, i.e. in a pressure increase of the fluid.

Also, the chamber may be sufficiently filled with fluid, i.e. gas and/or liquid, to result in a change of the pressure within the fluid, and for it to be detectable. For example, the fluid may be saline. A conceivable standard pressure is 300 mm Hg, though others are contemplated and possible.

By way of providing a self-contained/sealed chamber accommodating fluid, wherein a pressure sensor measures the pressure (absolute value and/or change of the pressure) of the fluid, kinking of the catheter can be detected by way of the (relative) pressure change (pressure increase) inside the chamber, e.g., of the fluid in the chamber. In case of a kink, the pressure in the fluid at the inner side of the kinked catheter wall will increase, and the pressure at the outer side of the kinked catheter wall, if measured by way of a chamber, will also increase.

According to the pressure sensor of the present disclosure, irrespective of the direction of the kink, a kink may lead to an increased pressure. As only an increase of the pressure (and no decrease) may be measured, the measurement and post-processing may be simplified.

The size of the region the pressure of which is to be detected may be selected by adjusting the size and location of the chamber. Hence, the region in which a pressure change may be detected may be enlarged by enlarging the “measurement chamber” (the chamber housing the fluid) compared to “direct” pressure measurements by means of piezoelectric elements embedded directly within the material of the catheter wall, for example, as less electronics and/or fewer pressure sensors are needed to cover the desired region in which a pressure change is to be monitored. As such, a single pressure sensor may be used to detect a pressure (change) along the length of the chamber in the axial direction of the catheter. In other words, a larger region can be kept under surveillance than by way of “direct” measurement. Put differently, by way of the chamber/fluid in the catheter wall, the field of measurement can be enlarged as needed and measurement of the pressure in the fluid (not directly in the material of the chamber wall) allows for “indirect” determination of a pressure change and, hence, of a kink of the catheter.

Evidently, the pressure measurement by means of at least one chamber comprising fluid may, in some embodiments, allow for efficient pressure measurement without (unduly) compromising accuracy, which may, in some embodiments, simplify the measurements and/or increases efficiency.

Kinking of the catheter may refer to any deformation of the catheter in the transverse direction of the catheter, in particular including kinking of the catheter during the procedure. Deformation and collapsing of the catheter often lead to an irreversible kink.

Specifically, kinking of the catheter may increase friction between the catheter wall and the vessel (environment) through which the catheter is being navigated during the procedure, in some embodiments. Increased friction could result in further damage, such as damage to the catheter wall or even the inside of the catheter, which could then directly and/or indirectly further negatively impact the patient.

Specifically, at least some embodiments of the present may be advantageous if the exact location of a kink of the catheter is not of primary interest, but rather knowing the fact whether there is a kink is sufficient.

A relative change of the pressure of the fluid may be determined, as a pressure change may be of primary interest. Absolute measurements pertaining to the pressure are not necessary, but are not excluded. Specifically, the pressure change is a (relative) pressure increase.

The chamber is self-contained/sealed off/isolated against the environment. Specifically, no fluid communication or pressure communication between the fluid accommodated in the chamber and the outside is considered. The catheter wall is sealed against the environment/outside (such as blood pressure and atmospheric pressure), so that (substantially) no communication (specifically as to the pressure) with the environment takes place via the catheter wall. In other words, the catheter wall is configured such that no communication between the chamber and the environment with respect to the ambient pressure impacts the pressure measurement. Hence, the pressure which is measured inside the chamber, e.g., the fluid pressure, is based on a pressure change resulting from the deformation of the self-contained chamber and, hence, the fluid housed in the self-contained chamber. Any effects resulting from pressure fluctuations outside the catheter or inside the interior lumen of the catheter, e.g., a change of the catheter environment, are not considered.

The chamber is provided separately (separated) from the interior lumen, i.e. is not identical to the interior lumen. The chamber may be substantially neighboured/adjacent to the interior lumen of the catheter. However, the location of the chamber does not need to be immediately next to the interior lumen. The interior lumen may be a cavity, optionally substantially empty (e.g. representing an aspiration channel) and/or for accommodating a guidewire, stent and/or helix.

The catheter may be an introducer sheath, a guiding sheath or a support catheter. The catheter may be configured for aspiration (e.g. thrombectomy catheter), fragmentation (e.g. atherectomy catheter) and/or removal of removable material, such as thrombi and emboli, from blood vessels.

Optionally, the catheter wall encapsulates the chamber. In other words, the chamber may be completely embedded and surrounded by the catheter wall. Hence, the chamber may be completely surrounded by material of the catheter wall. This may represent a particularly space-saving embodiment.

Also, optionally, the pressure sensor may be positioned at least partially within the chamber. Hence, in addition to the communication between the pressure sensor and the fluid, the pressure sensor is positioned, at least partially, within the chamber, so as to ensure minimal space is required. It is conceivable that the chamber has a sensor-part in which the sensor is located, the (main) chamber and the sensor-part thereof being in communication with each other, so that the sensor is responsive to a pressure change of the fluid in the chamber. The sensor and the fluid are in fluid communication. For example, the sensor may be larger than the transverse extension of the (main) chamber so that the sensor-part designates a larger part of the chamber in which the sensor is located.

Generally, the sensor may be provided at the proximal end of the chamber. Hence, the chamber may have a part called “sensor-part” at its proximal end. As explained, optionally, the sensor part may have a larger diameter than the remaining chamber for accommodating the sensor. Generally, it may be desired to reduce the cross-sectional extension of the chamber in the transverse direction, as this may otherwise deteriorate the stability of the catheter.

In some embodiments, the interface may connect to a wire. Further optionally, the wire may extend at least partially within the catheter wall. The wire may connect the interface to the control unit. Further optionally, the wire may extend at least partially within the catheter wall. This may provide for safe and reliable connection between the pressure sensor and the control unit. Also wireless connections to the control unit such as via infrared, Bluetooth, WLan etc. are conceivable. Generally, the interface may be wired and/or wireless.

Optionally, the chamber may have an elongate/oblong extension in the axial direction of the catheter. Optionally, a single chamber extends along the axial direction. A single oblong chamber along the axial direction may be included in the catheter. In other words, the predetermined length of the catheter along which the pressure change resulting from kinking of the catheter is to be detected, may be covered by way of a single sensor.

In some embodiments, the chamber at least partially extends parallel to the interior lumen of the catheter, which extends in the axial direction of the catheter. Providing a chamber along the axial direction, e.g., parallel to the interior lumen of the catheter, may, in some embodiments, allow for most efficient detection of kinking of the catheter.

In a cross-sectional view perpendicular to the axial direction of the catheter, the extension of the chamber may be less than one half, optionally less than one third, further optionally less than one fourth of the substantially circumferential extension of the catheter wall. This means that the chamber may be, along the catheter wall, only extending within one half, one third or one fourth of the circumference of the catheter wall.

As to the size of the chamber, the volume may be between about 3 to about 15 mm³, optionally between about 5 to about 10 mm³. The chamber may house about 3 to about 15 ml fluid, optionally between about 5 and about 10 ml. Alternatively or additionally, the length of a chamber in the axial direction may be between about 100 mm and about 300 mm, optionally between about 150 mm and about 250 mm, further optionally approx. 200 mm.

As the catheter may have a substantially circular cross-section, the catheter walls may be substantially cylindrical, and the chamber may extend, in a cross-sectional view perpendicular to the axial direction, within about 180°, optionally within about 60°, further optionally within about 450 of the circle representing a cross-section of the catheter wall. By way of limiting the circumferential extension of the chamber accordingly, the pressure may, in some embodiments, be detected more accurately, as less (circumferentially extending) space in the chamber possibly compensating for deformation caused by kinking of the catheter may be available. Put differently, by reducing the circumferential extension of the chamber, the pressure change resulting from kinking of the catheter at a corresponding transversal location may be expected to be more accurate, as the pressure change is not “diluted” (e.g., at least fully compensated) by allowing fluid in the chamber to escape to regions of the chamber which are less affected by kinking of the catheter.

Optionally, in a cross-sectional view perpendicular to the axial direction of the catheter, the area of the chamber is included in less than about 50%, optionally less than about 30%, further optionally less than about 10% of the catheter wall. In other words, the catheter wall may be, up to about 90%, optionally about 70%, further optionally about 50%, free from a chamber, when viewed in a transverse cross-section. Hence, the volume of the chamber may be reduced, in some embodiments so as to avoid undue increase of the diameter, specifically of the thickness of the catheter.

In a further optional embodiment, the catheter having a predetermined length may include a chamber located in the distal part of the chamber. Specifically, the chamber may be located in the distal half of the catheter, optionally in the distal third of the catheter. Often, bending/kinking of the catheter takes place in the distal part, specifically the 50% or 30% of the catheter at the distal side of the catheter.

Instead of a single self-contained chamber, a plurality of such chambers may be present, wherein each of these chambers is self-contained/sealed off. In other words, the plurality of mutually sealed off chambers may not be in fluid communication with each other. Hence, each of the plurality off chambers may have, like the chamber described above, a pressure sensor in fluid communication with the fluid comprised in the respective chamber. Accordingly, each of the plurality of chambers may be self-contained and accommodate a fluid, each of the chambers substantially neighbouring the interior lumen of the catheter and structured to be responsive to kinking of the catheter wall. Each of the plurality of chambers may comprise a pressure sensor in fluid communication with the fluid housed in the respective chamber. Hence, in some embodiments, the catheter may be configured to allow for measurement of a change of pressure in the respective chamber in response to kinking of the catheter. By providing multiple chambers, each sealed off from each other, the pressure change may be measured at various locations along the catheter wall independently.

Optionally, the chambers are positioned one after the other along the axial direction of the catheter and/or the catheter is free from a chamber provided opposite a chamber relative to the interior lumen. Possible embodiments pertaining to the arrangement of various chambers relate to the axial and transverse configuration. Specifically, in some embodiments, it may be sufficient to provide a chamber only at one side along the catheter wall, as such chamber equally allows for detection of all directions of a kink in a catheter to be detected, as the chamber and, consequently, the fluid housed in the chamber, is being compressed.

Optionally, the interior lumen of the catheter may be adjacent to two chambers at two (substantially radially) opposite sides of the interior lumen, when viewed in a cross-sectional view perpendicular to the axial direction of the catheter. In other words, the interior lumen of the catheter may have two proximate chambers at two (substantially radially) opposite sides of the interior lumen. Of course, if two chambers on opposite sides of the interior lumen in the axial direction are provided, the accuracy of the measurement may be increased in some embodiments, as, in the control unit, two separate and at least partially redundant pressure measurements can be performed.

The pressure sensor may be based on piezoresistive strain gauge, capacitive, electromagnetic, piezoelectric, strain-gauge, resonant sensor (resonant frequency), or potentiometric measurement. It is contemplated that a pressure change of 1 mm Hg may be detectable, i.e. resolved, by way of the sensor, and/or the pressure range being between 250 mm Hg and 400 mm Hg. The pressure sensor is, for example, a passive resonant sensor made of inductive copper spirals applied on a polyamide substrate.

The present disclosure also pertains to a catheter of the present disclosure and a control unit, e.g., a kit comprising a catheter of the invention and a control unit.

More specifically, the control unit of the system of the invention may include identification means for identifying whether the measured pressure (change) reaches or exceeds a predetermined threshold. For example, in some embodiments, a threshold may be predetermined such that, if the threshold is reached, no damage to the catheter or the patient is to be expected. Hence, the threshold can be set so as to avoid any potential damage, in some embodiments.

Optionally, the control unit is configured to alert the clinician if the threshold is reached or exceeded. Optical or acoustic alerts are conceivable, so as to inform the clinician that kinking of the catheter has reached a critical state. In other words, audible and/or visual warning signal may be issued to alert the clinician of a kink in the catheter. Alternatively or additionally, a value related to the pressure increase may be indicated, such as a relative increase of the pressure. This may, in some embodiments, allow the clinician to consider removal of the catheter being used during the current procedure and replacement of the catheter.

Optionally, the control unit may include monitoring means for monitoring the change of the measured pressure over time and for determining an origin of a pressure increase based on this change. Specifically, in some embodiments, it may be possible to differentiate between a tortuous anatomy, which leads to (strong) bending of the catheter, and a kink in the catheter. Specifically, a more abrupt increase in the pressure over time may be expected in case of a kink in the catheter, compared to a pressure increase resulting from bending of the catheter when navigating through tortuous anatomy. For example, only if the origin of compression of the fluid in the chamber is determined to be a kink in the catheter, an alert may be issued. Otherwise, if the catheter has been bent due to tortuous anatomy, no alert may be issued.

The disclosure is also directed to the following catheter system:

Catheter system comprising:
a control unit; and
a catheter comprising:
   a bendable catheter wall defining an interior lumen and one or more chambers embedded within the bendable catheter wall adjacent and fluidly isolated from the interior lumen; and
   one or more pressure sensors configured to output a signal indicative of a pressure within the one or more chambers,
wherein the control unit:
   recites/receives the signal from the one or more pressure sensors; and
   determines, based on the signal from the one or more pressure sensors, whether the catheter is kinked.

The catheter system may have the features of the dependent claims 2 to 17.

Also contemplated is a method for detecting a kink in a catheter as follows:

A method for detecting a kink in a catheter, the method comprising:
receiving with a control unit a pressure signal from one or more pressure sensors coupled to a catheter, the catheter comprising a bendable catheter wall defining an interior lumen and one or more chambers embedded within the bendable catheter wall adjacent and fluidly isolated from the interior lumen, wherein the one or more pressure sensors output a signal indicative of a pressure (change) within the one or more chambers;
determining that the catheter is kinked in response to a pressure signal above a predetermined threshold.

Optionally, the method may comprise outputting an alert with the control unit in response to determining the catheter is kinked.

Optional embodiments for this method are defined in the dependent claims 2 to 17.

The present disclosure will be described with respect to the appended drawings in the following.

### BRIEF DESCRIPTION OF THE DRAWINGS

FIG. 1a shows a cross-section of a catheter along the axial direction in an original, relaxed state, according to one or more embodiments shown and described herein;

FIG. 1b shows a cross-section of a catheter along the axial direction in the kinked state, according to one or more embodiments shown and described herein;

FIG. 1c shows a cross-section of a catheter along the axial direction in the original, relaxed state with indications of the locations of the chamber, according to one or more embodiments shown and described herein;

FIG. 2a shows a transverse cross-section of the catheter of FIG. 1a, according to one or more embodiments shown and described herein;

FIG. 2b shows an alternative transverse cross-section of the catheter of FIG. 1a, according to one or more embodiments shown and described herein;

FIG. 2c shows an alternative transverse cross-section of the catheter of FIG. 1a, according to one or more embodiments shown and described herein;

FIG. 3 shows an axial cross-section of a catheter, according to one or more embodiments shown and described herein; and

FIG. 4 shows a system of the disclosure comprising a control unit and a catheter, according to one or more embodiments shown and described herein.

### DETAILED DESCRIPTION

Embodiments of the disclosure will be described in the following with reference to the appended drawings.

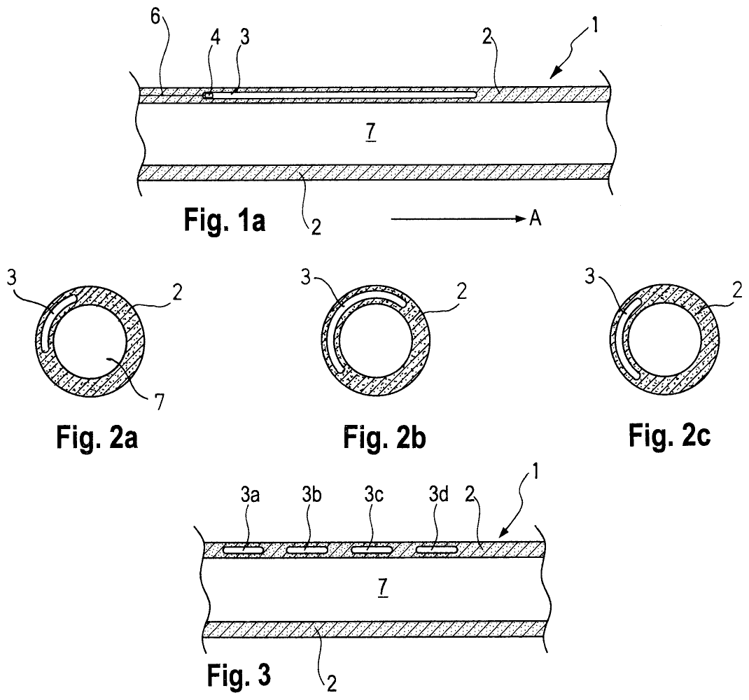

A catheter 1 comprising a bendable catheter wall 2 is shown in FIG. 1a. The catheter wall 2 defines a substantially central interior lumen 7 of the catheter 1. For example, the interior lumen 7 can receive a guidewire (not shown), a stent (not shown) for deployment, a helix (not shown) for a conveying clot removed from the vessel wall, etc. Various applications are conceivable.

The catheter wall 2 includes a self-contained, i.e. sealed chamber 3. The chamber 3 is delimited by a chamber wall. The chamber 3 accommodates fluid, i.e. liquid or gas. The pressure of the fluid changes upon compression of the volume of the chamber 3. Hence, deflection of a wall of the chamber 3, the deflection or deformation resulting from kinking of the catheter wall 2, results in a pressure change of the fluid housed in the chamber 3. In the embodiment of FIG. 1, the catheter wall 2 encapsulates the chamber 3. Accordingly, the catheter wall 2 constitutes the chamber wall.

The chamber 3 substantially neighbours the interior lumen 7. For example, the chamber 3 is displaced radially with respect to the central interior lumen 7 of the catheter 1. Hence, the chamber 3 is structured to be responsive to kinking of the catheter wall 2.

The catheter 1 comprises a pressure sensor 4, wherein the pressure sensor 4 is positioned within the chamber 3. The pressure sensor 4 is in communication with the fluid, so that the pressure sensor 4 is configured to measure a pressure of the fluid. The pressure sensor 4 may be a piezoelectric sensor, though other sensors are also contemplated and possible.

The pressure sensor 4 comprises an interface for communicating the measured pressure to a control unit 5 (not shown in FIGS. 1a, 1b to 3).

Thus, the catheter 1 is configured to allow for measurement of a change of the pressure in the fluid in response to kinking of the catheter 1. As such, the catheter wall 2 (and the chamber wall, if not identical to the catheter wall 2) is flexible so as to allow for bending.

As shown in FIG. 1a, the interface of the pressure sensor 4 connects to a wire 6. The wire 6 extends at least partially within the catheter wall 2 and along the catheter wall 2. For example, at the proximal end of the catheter 1, the wire 6 may exit the catheter wall 2 and connect to the control unit 5.

The chamber 3 may have an elongate, i.e. oblong extension in the axial direction A of the catheter 1. As shown in FIG. 1, a single chamber 3 may extend along the axial direction A. Here, the chamber 3 at least partially extends parallel to the interior lumen 7 of the catheter 1.

With continued reference to FIG. 1a, the chamber 3 may be located in a distal half of the catheter 1.

In FIG. 1a, the catheter 1 is in the original, e.g., relaxed, state. In other words, the catheter 1 (as well as the chamber 3) is straight and extends along the axial direction A.

In FIG. 1b, the catheter is in a kinked state. The kink K is present at angle (of approx. 90° in this example) formed by the catheter 1. Accordingly, the chamber 3 is deformed, which entails a compression of the fluid in the chamber 3 and a pressure increase of the fluid. More specifically, the chamber 3 is divided into two portions by way of the kink K: Two chamber portions 3-1 and 3-2 can be identified, which are separated by chamber walls which contact each other. In other words, as the chamber walls come closer to each other and approach each other when the catheter 1 is bent and at some stage kinked, the total volume in which the fluid is received is reduced, which in turn increases the pressure of the fluid in each of the chamber portions 3-1 and 3-2. The pressure sensor 4 measures the pressure in one of the chambers 3-1 and 3-2, depending on the location of the pressure sensor 4 in relation to the kink K.

Generally, the catheter (including the chamber wall) may be made of polyamide.

In a specific example, the catheter wall 2 has a thickness of 0.3 mm and is made of polyamide. The chamber 3 includes an enlarged sensor-part 3a for the sensor 4. This is indicated in FIG. 1c showing a catheter 1 in the straight configuration. The sensor 4 is located in the sensor part 3a of the chamber 3 at the proximal end of the chamber 3. In one example, the chamber 3 in which a kink is detectable (and supposed to occur) has a volume of 6.2 mm³ and the volume of the sensor-part 3a is 0.3 mm³. The fluid contained in the chamber is saline (volume of fluid is 6.5 mL and the standard pressure is 300 mm Hg). The length of the chamber 3 is 200 mm, and the chamber 3 is surrounded by material of the catheter wall of at least a thickness of 0.05 mm. The sensor-part 3a is in fluid communication with the other parts of the chamber, but has a larger cross-section for accommodating the (relatively large) sensor 4. In a cross-section along the axial direction, the area of the (main) chamber 3 is about 10 mm², while the area of the sensor-cavity 3a is about 0.2 mm². The pressure sensor 4 is a passive resonant sensor made of inductive copper spirals applied on a polyamide substrate and a pressure increase of 1 mm Hg is detectable. With reference to FIG. 1b above, it is noted that the sensor part 3a, i.e. the sensor 4, may be in fluid communication with only one of the chamber regions 3-1 and 3-2 in the kinked state, and not in fluid communication of the entire chamber 3 in the event of a kink.

A kink may be identified based on the pressure change per time. For example, a threshold may be 3 mm Hg pressure difference within 0.5 seconds, i.e. 3 mm Hg/0.5 s=6 mm Hg/s. Generally, a threshold may be between 4 and 8 mm Hg/s. This means that a pressure change of less than the threshold (here: 3 mm Hg pressure within 0.5 seconds) is not related to a kink, as it may originate from tortuous anatomy, whilst a pressure change exceeding the threshold (here: at least 3 mm Hg pressure within 0.5 seconds) is considered to originate from a kink.

FIG. 1b reflects that a kink K is detectable even if the chamber 3 is on an outer side O of the kink K, while the catheter wall 2 at the inner side I of the kink K has collapsed. The outer side O of the kink K includes an angle of about 90° between the catheter walls. On the inner side I, the angle is even more acute (i.e. smaller), as the collapse of the catheter walls 2 includes an inward pleat/fold at the kink K. Evidently, if a kink K occurs at the side of the catheter 1 at which the chamber 3 is located (i.e. if the catheter of FIG. 1b was not angled to the left side as shown, but to the right side), the chamber 3 would be affected as well, presumably even stronger than by the kink K shown in FIG. 1b.

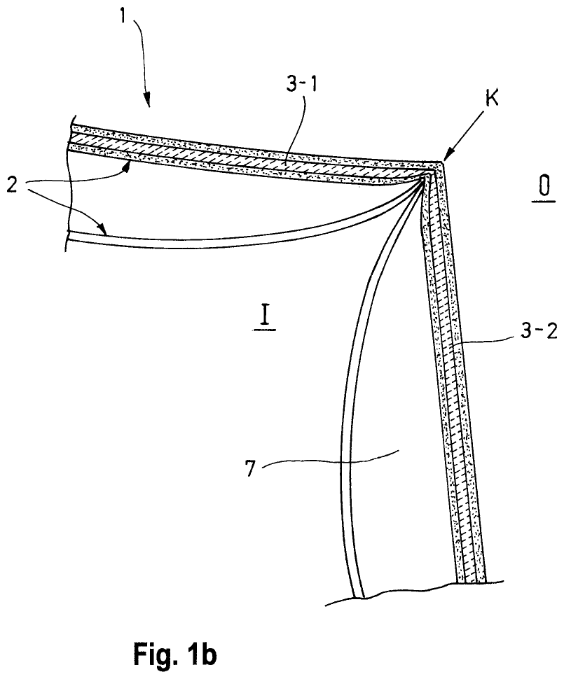

Turning to FIG. 2a to 2c, cross-sectional views perpendicular to the axial direction A, i.e. in the transverse direction, are shown. The chamber 3 is embedded in the circular configuration defined by the catheter wall 2 in the cross-sectional view. In FIG. 2a, the extension of the chamber 3 is less than one fourth of the substantially circumferential extension of the catheter wall 2. In the alterative FIG. 2b, the extension of the chamber 3 is less than one half of the substantially circumferential extension of the catheter wall 2. In the further alternative FIG. 2c, the extension of the chamber 3 is less than one third of the substantially circumferential extension of the catheter wall 2.

With continued reference to FIG. 2a/2b/2c, the area of the chamber 3 is included in less than one fourth/half/third of the area of the catheter wall 2 in the transverse cross-section.

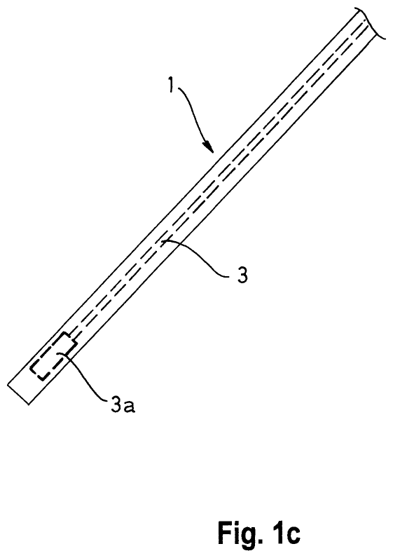

FIG. 3 shows an embodiment having a plurality of chambers 3a to 3d. Each of the plurality of chambers 3a to 3d is mutually sealed off. The chambers 3a to 3d are compatible with the chamber 3 shown in FIG. 1, and may be provided additionally or alternatively. In other words, a combination of the embodiments of FIGS. 1 and 3 is conceivable.

The chambers 3a to 3d as shown in FIG. 3 are positioned one after the other along the axial direction A of the catheter 1. In the axial cross-section shown in FIG. 3, the catheter wall 2 opposite the plurality of chambers 3a to 3d is free from chambers. However, in the embodiments shown in FIGS. 1, 2 and 3, for example, corresponding opposite chambers at the opposite side of the interior lumen 7 of the catheter may additionally be provided.

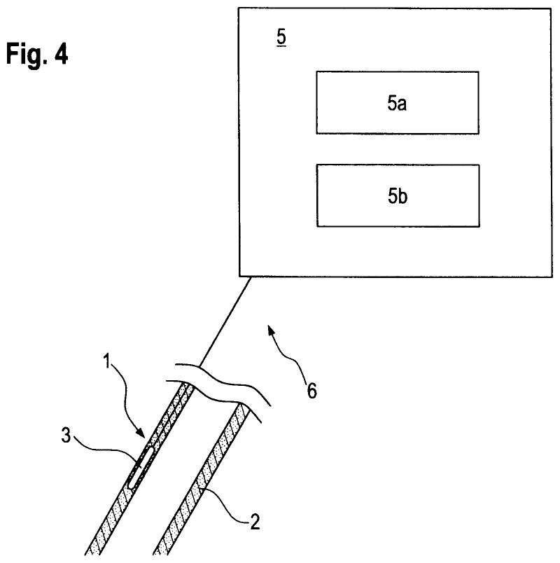

FIG. 4 reflects a system comprising a catheter 1 and a control unit 5. The control unit 5 comprises identification means 5a for identifying if the measured pressure reaches or exceeds a predetermined threshold and/or if the pressure change results from a kink K in the catheter 1, i.e. for determining an origin of the pressure increase based on said change. The predetermined threshold may be about 400 mm Hg, about 350 mm Hg or about 300 mm Hg. If the predetermined pressure threshold is exceeded and the identification means 5a determines a kink to be present, the identification unit issues an alert. The alert may be given via a user interface and/or as audible, visual, and/or haptic feedback. By doing so, the identification means 5a alerts the clinician that the threshold is reached or exceeded.

For a preceding step, the control unit 5 includes monitoring means 5b for monitoring the change of the measured pressure over time. In some embodiments, the monitoring means 5b may (temporarily) store measured pressure values at predetermined time intervals, such as every 1 or 5 seconds and/or provide the measured pressure as a function of time (such as every 1 or 5 seconds).

The monitoring means 5b and the identification means 5a may be a common, single unit and may share components, in the control unit 5.

For detecting a kink K in a catheter 1, the following steps may be performed: In the control unit 5, a pressure signal from one or more pressure sensors 4 coupled to the catheter 1 is received, the catheter 1 comprising a bendable catheter wall 2 defining an interior lumen 7 and one or more chambers 3 embedded within the bendable catheter wall 2 adjacent and fluidly isolated from the interior lumen 7, wherein the one or more pressure sensors 4 output a signal indicative of a pressure (change) within the one or more chambers 3. The user determines that the catheter 1 is kinked in response to a pressure signal above a predetermined threshold. An alert with the control unit in response to determining the catheter is kinked may be output.

1. A catheter comprising a bendable catheter wall defining an interior lumen of the catheter,
the catheter wall including a sealed chamber accommodating fluid, the chamber separate from the interior lumen and structured to be deformable in response to kinking of at least a part of the catheter wall,
the catheter further comprising a pressure sensor in communication with the fluid, the pressure sensor configured to measure a pressure of the fluid,
the pressure sensor further comprising an interface for communicating the measured pressure to a control unit,
the catheter configured such that kinking of at least a part of the catheter wall is detectable by way of a pressure increase in the fluid in the chamber.

2. The catheter of claim 1, wherein the catheter wall encapsulates the chamber.

3. The catheter of claim 1, wherein the pressure sensor is positioned at least partially within the chamber.

4. The catheter of claim 1, wherein the interface connects to a wire for connection to the control unit, optionally the wire extending at least partially within the catheter wall.

5. The catheter of claim 1, wherein the chamber has an elongate extension in an axial direction of the catheter, optionally a single chamber extending along the axial direction.

6. The catheter of claim 1, wherein the chamber at least partially extends parallel to the interior lumen of the catheter.

7. The catheter of claim 1, wherein in a cross-sectional view perpendicular to an axial direction, the extension of the chamber is less than one half, optionally one third, further optionally one fourth of the substantially circumferential extension of the catheter wall.

8. The catheter of claim 1, wherein
in a cross-sectional view perpendicular to an axial direction, the area of the chamber is included in less than 50%, optionally less than 30%, further optionally less than 10% of the area of catheter wall.

9. The catheter of claim 1, wherein the catheter has a predetermined length and the chamber is located in the distal half of the catheter, optionally the distal third of the catheter.

10. The catheter of claim 1, wherein the catheter comprises a plurality of mutually sealed-off chambers (3a-d).

11. The catheter of claim 10, wherein the chambers are positioned one after the other along an axial direction of the catheter and/or the catheter is free from a chamber provided opposite a chamber relative to the interior lumen.

12. The catheter of claim 10, wherein, in a cross-sectional view perpendicular to an axial direction, the interior lumen of the catheter is adjacent to two chambers at two opposite sides of the interior lumen.

13. The catheter of claim 1, wherein the pressure sensor is based on piezoresistive strain gauge, capacitive, electromagnetic, piezoelectric, strain-gauge, resonant sensor, and/or potentiometric measurement.

14. A system comprising the catheter of claim 1 and the control unit.

15. The system according to claim 14, the control unit including identification means for identifying whether the measured pressure and/or pressure change reaches or exceeds a predetermined threshold.

16. The system according to claim 14, wherein the control unit is configured to alert the clinician if the threshold is reached or exceeded.

17. The system claim 14, wherein the control unit includes monitoring means configured for monitoring the change of the measured pressure over time and for determining an origin of a pressure increase based on said change.
